(19) United States
(12) Patent Application Publication
(10) Pub. No.: US 2020/0376017 A1
(43) Pub. Date: Dec. 3, 2020

(54) AMORPHOUS ERTUGLIFLOZIN AND PROCESS FOR ITS PREPARATION

(71) Applicant: CADILA HEALTHCARE LIMITED, Ahmedabad (IN)

(72) Inventors: Sanjay Jagdish DESAI, Ahmedabad-Gujarat (IN); Tadikonda PRATAP V., Ahmedabad-Gujarat (IN); Mahesh Laljibhai RUPAPARA, Ahmedabad-Gujarat (IN); Kumar Kamlesh SINGH, Ahmedabad-Gujarat (IN); Hardik Bhikhubhai GHODASARA, Ahmedabad-Gujarat (IN)

(73) Assignee: CADILA HEALTHCARE LIMITED, Ahmedabad (IN)

(21) Appl. No.: 16/887,214

(22) Filed: May 29, 2020

(30) Foreign Application Priority Data
May 31, 2019 (IN) 201921021737
Jul. 22, 2019 (IN) 201921029539
Dec. 31, 2019 (IN) 201921054693
Mar. 30, 2020 (IN) 202021013852

(51) Int. Cl. A61K 31/7048; A61K 9/10

(52) U.S. Cl. CPC A61K 31/7048; A61K 9/10

(57) ABSTRACT

An amorphous form of ertugliflozin and process for its preparation is described. A solid form of ertugliflozin and process for preparation thereof is also described.

### FIELD OF THE INVENTION

The invention relates to amorphous form of ertugliflozin and process for its preparation. In particular, the invention relates to solid form of ertugliflozin and process for preparation thereof. More particularly, the invention relates to ertugliflozin, amorphous form of ertugliflozin and the process for preparation thereof.

### CROSS REFERENCE APPLICATIONS

This application is a cognate complete specification claiming priority from co-pending Indian patent applications—201921021737 filed on 31 May 2019, 201921029539 filed on 22 Jul. 2019, 201921054693 filed on 31 Dec. 2019 and 202021013852 filed on 30 Mar. 2020; all of which are incorporated herein by reference.

### BACKGROUND OF THE INVENTION

The following discussion of background is intended to present the invention in an appropriate technical context and allow its significance to be properly appreciated.

Ertugliflozin is a sodium glucose co-transporter 2 (SGLT2) inhibitor indicated for use in type 2 diabetes mellitus. Chemical name for ertugliflozin is (1S,2S,3S,4R,5S)-5-(4-chloro-3-(4-ethoxybenzyl)phenyl)-1-(hydroxymethyl)-6,8-dioxabicyclo [3.2.1]octane-2,3,4-triol and its chemical structure can be represented as below

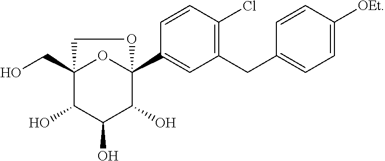 [SMILES: CCOc1ccc(Cc2cc([C@]34OC[C@](CO)(O3)[C@@H](O)[C@H](O)[C@H]4O)ccc2Cl)cc1]

International (PCT) Publication Nos. WO 2003/099836, WO 2010/023594, WO 2012/019496, WO 2014/187365, WO 2014/206349, WO 2014/159151, WO 2015/043473, WO 2016/189463 & WO 2018/055496; and Org. Process Res. Dev. 2014, Vol. 18(1) Pg. 57-65 and Org. Process Res. Dev. 2014, Vol. 18(1), Pg. 66-81 discuss one or more of ertugliflozin, process for preparation of ertugliflozin, process for the preparation of ertugliflozin intermediates, or precursors, or solid forms of ertugliflozin, or process for preparation thereof.

These publications discuss that despite extensive screening, a crystalline free form of ertugliflozin has not been identified. It exists as a hygroscopic amorphous solid with a low glass transition temperature, which is inappropriate for the clinical development. There exists a need to provide free form of ertugliflozin in a solid form appropriate for the clinical development. The inventors of the present invention have developed an amorphous form of ertugliflozin, which is suitable for pharmaceutical use.

### SUMMARY OF THE INVENTION

In one general aspect, the present invention provides an amorphous form of ertugliflozin. In another general aspect, the present invention provides a stable amorphous form of ertugliflozin.

In another general aspect, the present invention provides an amorphous solid dispersion comprising ertugliflozin.

In another general aspect, the present invention provides process for the preparation of amorphous ertugliflozin.

In another general aspect, the present invention provides process for the preparation of stable amorphous form of ertugliflozin.

In another general aspect, the present invention provides process for the preparation of amorphous solid dispersion of ertugliflozin.

In another general aspect, the present invention provides a composition comprising amorphous ertugliflozin, stable amorphous ertugliflozin or amorphous solid dispersion of ertugliflozin and pharmaceutically acceptable carrier.

In another general aspect, the present invention provides compounds A to G as described in Table 1.

In another general aspect, the present invention provides a process for preparation of ertugliflozin free from one or more of compounds A to G.

In another general aspect, the present invention provides a composition comprising ertugliflozin and one or more of compounds A to G.

In another general aspect, the present invention provides a composition comprising ertugliflozin or co-crystal thereof or amorphous solid dispersion thereof, and one or more of compound selected from F and G.

### BRIEF DESCRIPTION OF DRAWINGS

FIG. 1: Powder X-ray diffraction (XRD) pattern of amorphous solid dispersion of ertugliflozin and HPMC (1:1).

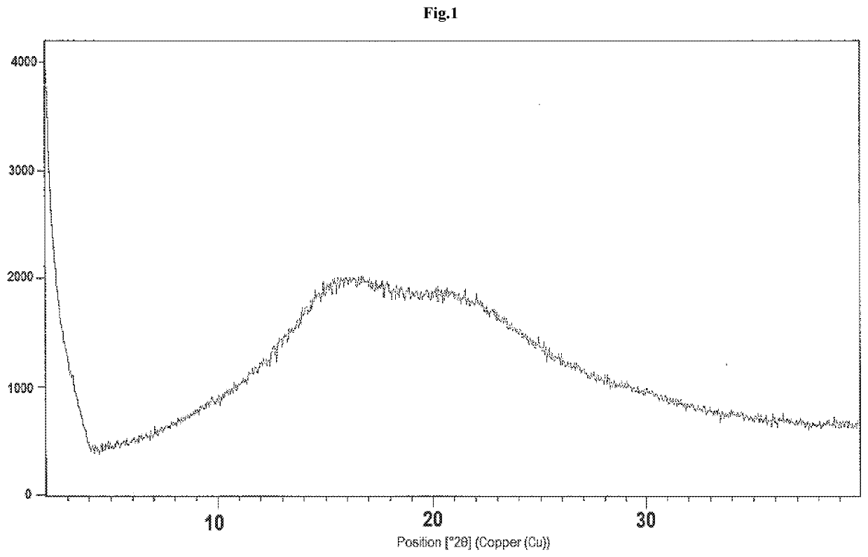

FIG. 2: Powder X-ray diffraction (XRD) pattern of amorphous ertugliflozin.

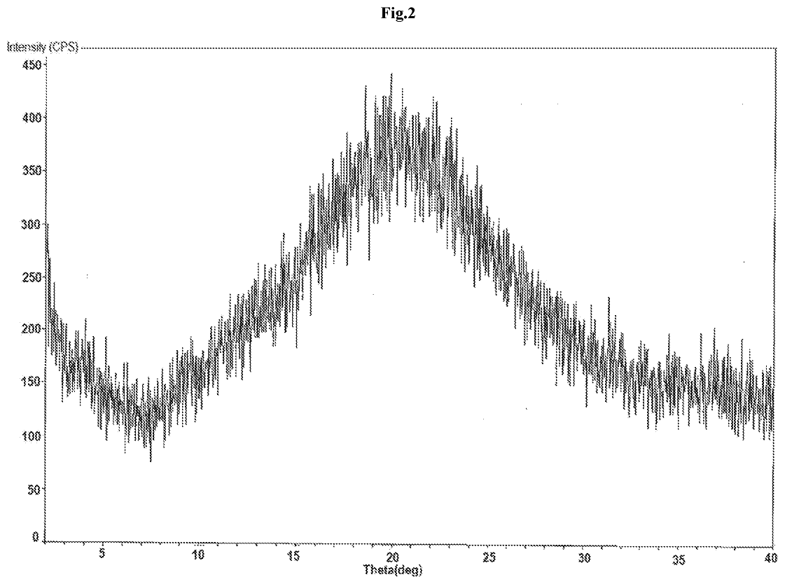

FIG. 3: Powder X-ray diffraction (XRD) pattern of amorphous solid dispersion of ertugliflozin with copovidone (1:1).

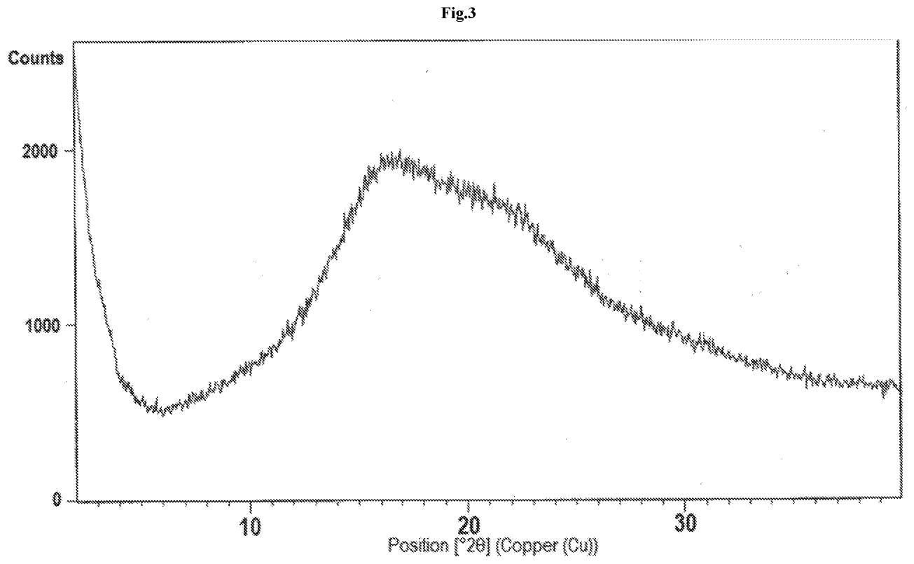

FIG. 4: Powder X-ray diffraction (XRD) pattern of amorphous solid dispersion of ertugliflozin with copovidone (1:1) after 3 months at 40° C. & 75% RH.

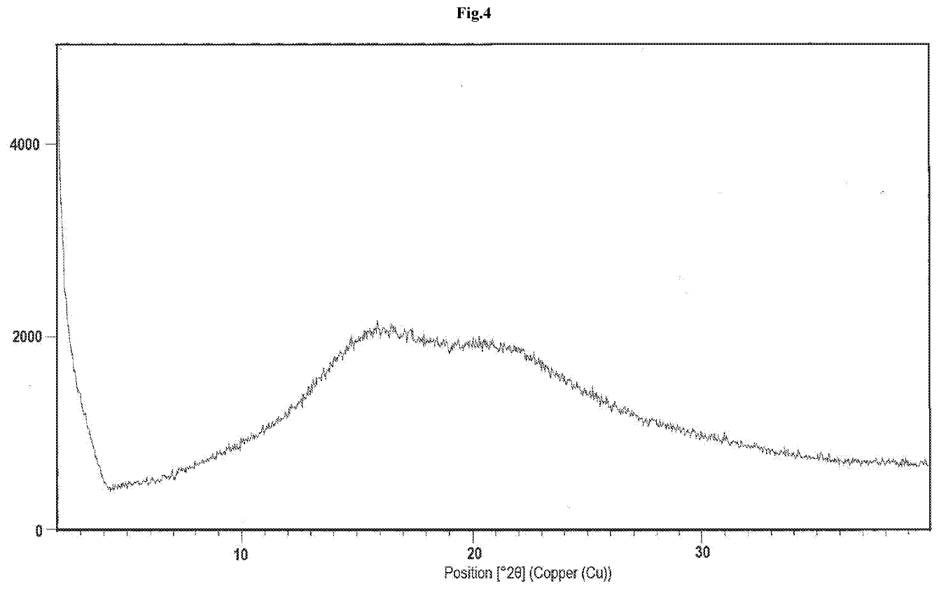

### DETAILED DESCRIPTION OF THE INVENTION

The invention can further be understood in light of the description of exemplary aspects provided herein after. It is to be understood that the description, in no way, is intended to limit the scope of the invention to the specifically described aspects only. The equivalents and variants thereof, apparently obvious to those skilled in the art, are also included within the scope of the present invention.

Detailed description of routine and conventional unit operations, which are easily understood by the skilled artisan, are not included herein. Such routine unit operations are to be construed so as ordinarily understood and as routinely practiced by the person skilled in the field of the invention, unless otherwise specifically described.

The term “solution” as used herein, unless described otherwise, does not necessarily mean only a clear solution or the one wherein the solute is completely soluble in the solvent; all the intermediate phases of mixture of components, starting from a state wherein the solute has just started getting dissolved in the solvent to a state wherein the solute has completely dissolved in the solvent, are also included within the expression ‘solution’.

The ranges recited herein also include the values denoted as the limits thereof. The numerical values recited as the limits are not to be construed as absolute values. Any value outside the recited ranges, wherein the difference between the values is insignificant considering the nature or the property of the variable to which the limit is applied, including any analytical variation in measuring those values, are also considered to be included within those ranges.

As used herein, the term “area percentage of HPLC” refers to the area percentage of a peak in HPLC chromatogram. In one aspect, the area percentage is relative to the total area of the composition in an HPLC chromatogram; and in another aspect, the area percentage is relative to the area of ertugliflozin in a HPLC chromatogram.

Undissolved solid and/or foreign particles, if any, can be removed before solid formation and/or solvent removal. A suitable technique useful for removal of solids can be selected from, but not limited to, filtration, decantation and centrifugation.

The product(s) obtained may further be converted to any other physical forms thereof which includes but not specifically limited to salt(s), solvate(s), hydrate(s), co-crystal(s) and solid dispersion(s) in either crystalline or amorphous forms and/or subjected to further physical processing like milling, shifting or other suitable powder processing techniques to adjust the particle size of the product to desired levels.

The term “pharmaceutically acceptable” as used herein means useful in preparing a pharmaceutical composition that is generally non-toxic and is not biologically undesirable, and is acceptable for veterinary or human pharmaceutical use.

The term “composition” as used herein means a physical mixture of two or more components.

The term “pharmaceutical composition” as used herein means a drug product comprising the active ingredient(s) & pharmaceutically acceptable excipient(s), as well as any product, which results, directly or indirectly, from combination, complexation or aggregation of any two or more of the ingredients.

In one general aspect, the present invention provides an amorphous form of ertugliflozin.

In another general aspect, the present invention provides a stable amorphous form of ertugliflozin.

In another general aspect, the present invention provides an amorphous solid dispersion comprising ertugliflozin.

In another general aspect, the present invention provides an amorphous solid dispersion comprising ertugliflozin and a polymer.

The polymer can be a pharmaceutically acceptable polymer. In general, the polymer can be an ionic- or a non-ionic-polymer. The polymer can be selected from a group comprising hydroxypropylmethyl cellulose (HPMC), hydroxypropylmethyl cellulose acetate succinate (HPMC-AS) and polyvinyl pyrrolidone (PVP). In general, different grades of PVP selected from, but not limited to, K-15, K-30, K-60, K-90 and K-120, can be used.

The polymer can be a single polymer or a mixture of plurality of polymers.

In another general aspect, the present invention provides an amorphous solid dispersion comprising ertugliflozin and hydroxypropylmethyl cellulose (HPMC).

In another general aspect, the present invention provides an amorphous solid dispersion comprising ertugliflozin and hydroxypropylmethyl cellulose acetate succinate (HPMC-AS).

In another general aspect, the present invention provides an amorphous solid dispersion comprising ertugliflozin and polyvinyl pyrrolidone (PVP).

Ertugliflozin and the polymer can be present in the solid dispersion in a ratio, by weight, from 9:1 to 1:9; preferably from 4:1 to 1:4; more preferably from 3:2 to 2:3; and most preferably from 1.2:1 to 1:1.2.

In another general aspect, the present invention provides an amorphous solid dispersion comprising ertugliflozin and a polymer in a ratio, by weight, from 9:1 to 1:9; preferably from 4:1 to 1:4; more preferably from 3:2 to 2:3; and most preferably from 1.2:1 to 1:1.2.

In another general aspect, the present invention provides an amorphous solid dispersion comprising ertugliflozin and a polymer in a ratio, by weight, from 1.2:1 to 1:1.2.

The amorphous solid dispersion of ertugliflozin is an amorphous form of ertugliflozin suitable for preparation of pharmaceutically acceptable dosage form.

The amorphous solid dispersion of ertugliflozin and the polymer provides better solubility and dissolution profile for ertugliflozin compared to physical mixture of ertugliflozin and the polymer.

The amorphous solid dispersion of ertugliflozin is an amorphous form of ertugliflozin appropriate for clinical development.

The amorphous solid dispersion of ertugliflozin is a stable amorphous form of ertugliflozin.

The amorphous solid dispersion of ertugliflozin is an amorphous form of ertugliflozin, which remains physically and chemically stable during drying and storage.

In another general aspect, the present invention provides a stable amorphous form of ertugliflozin.

In another general aspect, the present invention provides an amorphous form of ertugliflozin, which remains physically and chemically stable during drying and storage.

In another general aspect, the present invention provides a stable amorphous form of ertugliflozin, which contains less than 0.5% (wt/wt) of total impurities after exposure to 75% RH at 40° C. for at least 3 months.

In another general aspect, the present invention provides a stable amorphous form of ertugliflozin, which does not convert to any other solid form after exposure to 75% RH at 40° C. for at least 3 months.

In another general aspect, the present invention provides an amorphous solid dispersion comprising ertugliflozin, wherein ertugliflozin contains less than 0.5% (wt/wt) of total impurities after exposure to 75% RH at 40° C. for at least 3 months.

In another general aspect, the present invention provides an amorphous solid dispersion comprising ertugliflozin, wherein ertugliflozin does not convert to any other solid form after exposure to 75% RH at 40° C. for at least 3 months.

In another general aspect, the present invention provides a pharmaceutical composition comprising amorphous ertugliflozin and pharmaceutically acceptable carrier.

In another general aspect, the present invention provides a pharmaceutical composition comprising stable amorphous ertugliflozin and pharmaceutically acceptable carrier.

In another general aspect, the present invention provides a pharmaceutical composition comprising amorphous solid dispersion of ertugliflozin and pharmaceutically acceptable carrier.

In another general aspect, the present invention provides a pharmaceutical composition comprising ertugliflozin, wherein ertugliflozin contains less than 0.5% (wt/wt) of total impurities after exposure to 75% RH at 40° C. for at least 3 months.

In another general aspect, the present invention provides a process for the preparation of ertugliflozin comprising:
(a) reacting a compound of Formula IX and a compound of Formula VIII to obtain a compound of Formula VII;

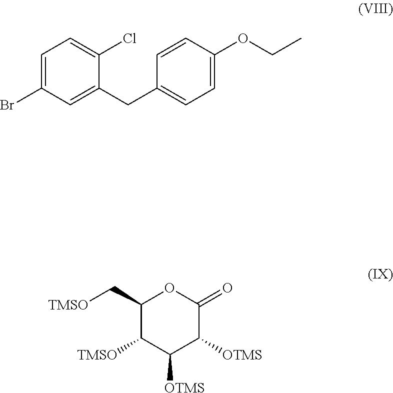

(b) converting the compound of Formula VII to a compound of Formula VII-T;

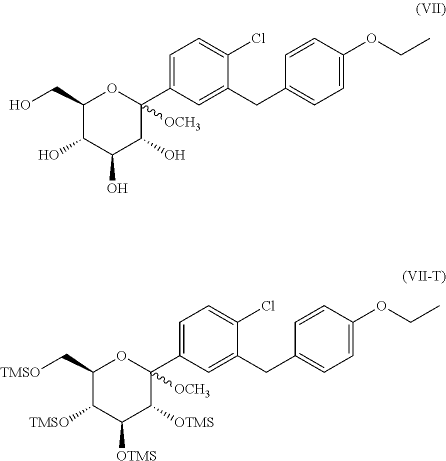

(c) converting the compound of Formula VII-T to a compound of Formula VI;
(d) converting the compound of Formula VI to a compound of Formula V;

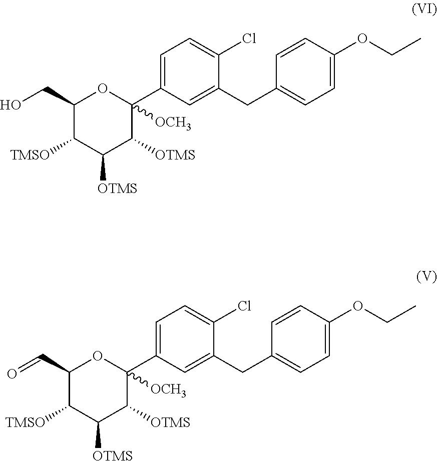

(e) converting the compound of Formula V to a compound of Formula IV; and

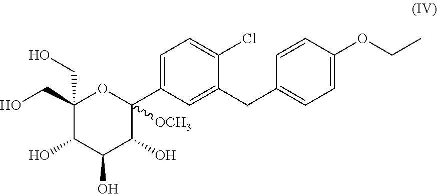

(f) converting the compound of Formula IV to ertugliflozin.

In another general aspect, the present invention provides a process for the preparation of ertugliflozin as depicted in Scheme-1.

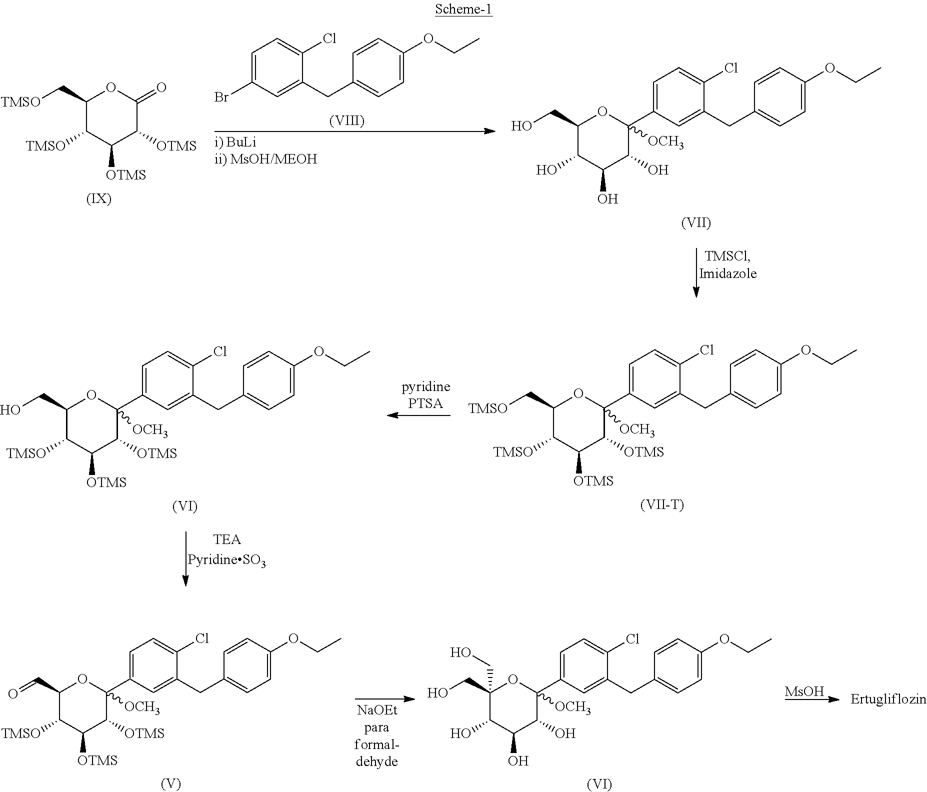

In another general aspect, the present invention provides a process for the preparation of ertugliflozin comprising:
(a) reacting a compound of Formula IX and a compound of Formula VIII in the presence of nBuLi followed by methanesulfonic acid in methanol to obtain a compound of Formula VII;
(b) reacting the compound of Formula VII with Trimethylsilyl chloride in the presence of imidazole to obtain a compound of Formula VII-T;
(c) treating the compound of Formula VII-T with pyridine and toluenesulfonic acid to obtain a compound of Formula VI;
(d) treating the compound of Formula VI with pyridine-sulfurtrioxide complex in the presence of triethylamine to obtain a compound of Formula V;
(e) reacting the compound of Formula V with sodium ethoxide and paraformaldehyde to obtain a compound of Formula IV; and
(f) treating the compound of Formula IV with methanesulfonic acid to obtain ertugliflozin.

Ertugliflozin can be directly obtained in its amorphous form, stable amorphous form or an amorphous solid dispersion at the end of the synthetic process; or it can be obtained as a residue or in any other physical/solid form and can be converted to amorphous form of ertugliflozin, or stable amorphous form of ertugliflozin or amorphous solid dispersion of ertugliflozin, directly from the residue or by preparing another solid form of ertugliflozin as an intermediate.

In another general aspect, the present invention provides a process for the preparation of amorphous ertugliflozin, stable amorphous form of ertugliflozin and amorphous solid dispersion of ertugliflozin.

In another general aspect, the present invention provides a process for the preparation of amorphous ertugliflozin comprising removal of the solvent from a solution comprising ertugliflozin.

In another general aspect, the present invention provides a process for the preparation of stable amorphous form of ertugliflozin comprising removal of the solvent from a solution comprising ertugliflozin.

In another general aspect, the present invention provides a process for the preparation of amorphous solid dispersion of ertugliflozin comprising removal of the solvent from a solution comprising ertugliflozin and a polymer.

In general, the solvent can be removed from the solution comprising ertugliflozin, or from the solution comprising ertugliflozin and a polymer, by one or more of distillation, distillation at reduced pressure, spray drying or lyophilisation. Any of the methods can be used for partial or complete removal of the solvent. Any combination of plurality of the methods can also be used in any suitable order for partial or complete removal of the solvent. The solid obtained in a mixture or suspension after partial removal of solvent can be isolated by filtration, filtration under vacuum, decantation, or centrifugation.

In another general aspect, the present invention provides a process for the preparation of amorphous solid dispersion of ertugliflozin comprising removal of the solvent from the solution comprising ertugliflozin and a polymer by distillation.

In another general aspect, the present invention provides a process for the preparation of amorphous solid dispersion of ertugliflozin comprising removal of the solvent from the solution comprising ertugliflozin and a polymer by distillation at reduced pressure.

In another general aspect, the present invention provides a process for the preparation of amorphous solid dispersion of ertugliflozin comprising removal of the solvent from the solution comprising ertugliflozin and a polymer by spray drying.

In another general aspect, the present invention provides a process for the preparation of amorphous solid dispersion of ertugliflozin comprising removal of the solvent from the solution comprising ertugliflozin and a polymer by lyophilization.

In general, the solution of ertugliflozin and the polymer can be prepared in one or more solvents selected from a group comprising water, alcohol, glycol, polyol, ketone and halogenated hydrocarbon.

In general, the alcohol can be selected from a group comprising methanol, ethanol, propanol, isopropanol, butanol and t-butanol; the glycol can be selected from a group comprising ethylene glycol and propylene glycol; the polyol can be selected from a group comprising glycerol, diethylene glycol and polyethylene glycol; the ketone can be selected from a group comprising acetone, methylethylketone and methylisobutylketone; and the halogenated hydrocarbon can be selected from a group comprising methylenedichloride and ethylenedichloride.

More than one solvent from the same or different generic groups as described herein above can also be used.

In general, ertugliflozin and the polymer are stirred in the solvent to obtain a solution and the solvent, after an optional filtration to remove foreign particles, is removed to obtain the amorphous solid dispersion of ertugliflozin.

In another general aspect, the present invention provides a process for the preparation amorphous solid dispersion of ertugliflozin comprising:
(a) dissolving ertugliflozin and a polymer in one or more solvents to obtain a solution; and
(b) removing the solvent from the solution to obtain the amorphous solid dispersion of ertugliflozin.

In another general aspect, the present invention provides a process for the preparation amorphous solid dispersion of ertugliflozin comprising:
(a) dissolving ertugliflozin and a polymer in one or more of water, alcohol, glycol, polyol, ketone or halogenated hydrocarbon to obtain a solution; and
(b) removing the solvent from the solution to obtain the amorphous solid dispersion of ertugliflozin.

In another general aspect, the present invention provides a process for the preparation amorphous solid dispersion of ertugliflozin comprising:
(a) dissolving ertugliflozin and hydroxypropylmethyl cellulose (HPMC) in one or more of water, alcohol, glycol, polyol or halogenated hydrocarbon to obtain a solution; and
(b) removing the solvent from the solution to obtain the amorphous solid dispersion of ertugliflozin.

In another general aspect, the present invention provides a process for the preparation of amorphous solid dispersion of ertugliflozin comprising:
(a) dissolving ertugliflozin and hydroxypropylmethyl cellulose acetate succinate (HPMC-AS) in one or more of alcohol, ketone or halogenated hydrocarbon to obtain a solution; and
(b) removing the solvent from the solution to obtain amorphous solid dispersion of ertugliflozin.

In another general aspect, the present invention provides a process for the preparation amorphous solid dispersion of ertugliflozin comprising:
(a) dissolving ertugliflozin and polyvinyl pyrrolidone (PVP) in one or more of water, alcohol, glycol, polyol or halogenated hydrocarbon to obtain a solution; and
(b) removing the solvent from the solution to obtain amorphous solid dispersion of ertugliflozin.

In another general aspect, the present invention provides a process for the preparation of amorphous ertugliflozin, stable amorphous form of ertugliflozin and amorphous solid dispersion of ertugliflozin; the process comprising:
(a) reacting a compound of Formula IX and a compound of Formula VIII to obtain a compound of Formula VII;
(b) converting the compound of Formula VII to a compound of Formula VII-T;
(c) converting the compound of Formula VII-T to a compound of Formula VI;
(d) converting the compound of Formula VI to a compound of Formula V;
(e) converting the compound of Formula V to a compound of Formula IV;
(f) converting the compound of Formula IV to amorphous ertugliflozin, stable amorphous form of ertugliflozin and amorphous solid dispersion of ertugliflozin.

In another general aspect, the present invention provides a process for the preparation of amorphous solid dispersion of ertugliflozin, the process comprising:
(a) reacting a compound of Formula IX and a compound of Formula VIII to obtain a compound of Formula VII;
(b) converting the compound of Formula VII to a compound of Formula VII-T;
(c) converting the compound of Formula VII-T to a compound of Formula VI;
(d) converting the compound of Formula VI to a compound of Formula V;
(e) converting the compound of Formula V to a compound of Formula IV;
(f) converting the compound of Formula IV to ertugliflozin;
(g) dissolving ertugliflozin and a polymer in one or more solvents to obtain a solution; and
(h) removing the solvent from the solution to obtain the amorphous solid dispersion of ertugliflozin.

In another general aspect, the present invention provides a stable amorphous form of ertugliflozin having purity of about 99.5% or more having total impurities 0.5% or less and single individual maximum impurity 0.15% or less as determined by area percentage of HPLC.

In another general aspect, the present invention provides an amorphous form of ertugliflozin having particle size distributions wherein the 10th volume percentile particle size (D10) is 50 μm or less, the 50th volume percentile particle size (D50) is 200 μm or less, or the 90th volume percentile particle size (D90) is 400 μm or less, or any combination thereof, as measured by Malvern Light Scattering method.

In another general aspect, the present invention provides an amorphous form of ertugliflozin having a chiral purity of about 95% or more, or about 98% or more, or about 99% or more, or about 99.5% or more, or about 99.8% or more, or about 99.9% or more, as determined using high performance liquid chromatography (HPLC).

In another general aspect, the present invention provides an amorphous form of ertugliflozin having a chemical purity of about 95% or more, or about 98% or more, or about 99% or more, or about 99.5% or more, or about 99.8% or more, or about 99.9% or more, as determined using high performance liquid chromatography (HPLC).

In another general aspect, the present invention provides compounds A to G as described in Table 1.

TABLE 1

| | |
|---|---|
| 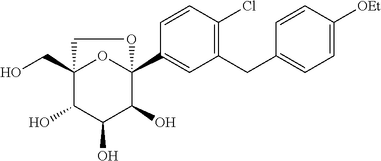 [SMILES: CCOc1ccc(Cc2cc([C@]34OC[C@](CO)(O3)[C@@H](O)[C@H](O)[C@@H]4O)ccc2Cl)cc1] | A |
| 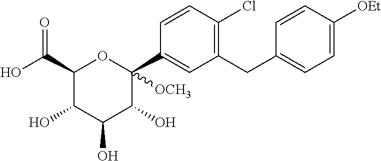 | B |
| 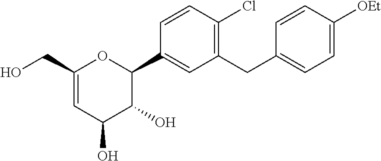 | C |
| 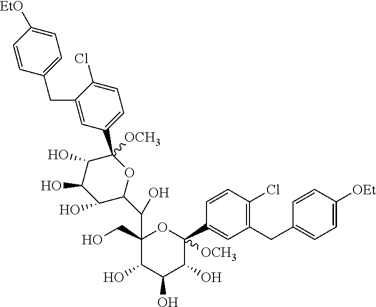 | D |
| 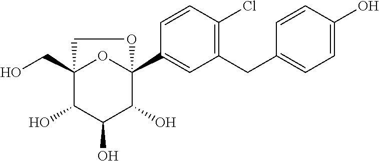 [SMILES: OC[C@@]12CO[C@@](c3ccc(Cl)c(Cc4ccc(O)cc4)c3)(O1)[C@H](O)[C@@H](O)[C@@H]2O] | E |
| 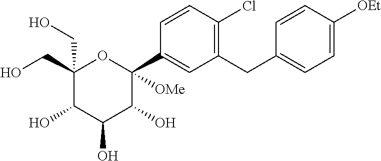 | F |
| 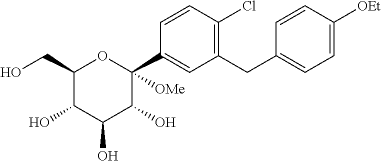 [SMILES: CCOc1ccc(Cc2cc([C@]3(OC)O[C@H](CO)[C@@H](O)[C@H](O)[C@H]3O)ccc2Cl)cc1] | G |

In another general aspect, the present invention provides an amorphous solid dispersion comprising ertugliflozin, wherein ertugliflozin has purity of about 99.5% or more, and one or more of compounds selected from A, B, C, D, E, F and G, less than about 0.15% each, as determined by area percentage of HPLC.

In another general aspect, the present invention provides a composition comprising 99.5% or more of ertugliflozin and one or more of compounds selected from A, B, C, D, E, F and G less than about 0.15% each, as determined by area percentage of HPLC.

In another general aspect, the present invention provides a composition comprising 99.5% or more of ertugliflozin and one or more of compounds selected from A, B, C, D, E, F and G less than about 0.15% each, as determined by area percentage of HPLC, wherein ertugliflozin is in amorphous form.

In another general aspect, the present invention provides a composition comprising ertugliflozin and one or more compounds selected from A to G.

In another general aspect, the present invention provides a composition comprising ertugliflozin and one or more of compound selected from F and G.

Another general aspect of the invention is a composition comprising amorphous solid dispersion of ertugliflozin and a pharmaceutically acceptable carrier.

The amorphous solid dispersion of ertugliflozin is further combined with pharmaceutically acceptable carrier to obtain a pharmaceutically acceptable composition.

In another general aspect, the present invention provides a pharmaceutical composition comprising an amorphous form of ertugliflozin or an amorphous solid dispersion comprising ertugliflozin, and one or more pharmaceutically acceptable carriers, excipients or diluents.

In another general aspect, the present invention provides a process for preparation of stable amorphous form of ertugliflozin comprising:
(a) placing an amorphous ertugliflozin under nitrogen atmosphere in a non-permeable bag and tied;
(b) placing the bag of step (a) inside another bag, optionally containing oxygen busters and sealing it;
(c) optionally placing the bag of step (b) inside a triple laminated bag, optionally containing oxygen busters and sealing it; and
(d) the sealed triple laminated bag inside a high density polyethylene (HDPE) container and sealing it.

The aspects of the invention can further be illustrated by following examples.

### EXAMPLES

### Example-1: Ertugliflozin

Step-1: Compound of Formula VII

In a 2 L round bottom flask, TMS protected glucarolactone (IX) (172 g) and 4-bromo-1-chloro-2-(4-ethoxybenzyl)benzene (VIII) (100 g) were stirred in dry tetrahydrofuran (800 mL) at −65 to −75° C. in an RBF. n-Butyl lithium (23% in hexane) (222 mL) was added and stirred for an hour. After the completion of the reaction, a solution of methane sulphonic acid (79.6 g) in methanol (750 mL) was added at (−25)-0° C. and the reaction mixture was warmed to room temperature and then stirred for 6 hours. The reaction mixture was quenched by aqueous sodium bicarbonate solution and stirred. The aqueous layer was washed with heptane and then extracted with dichloromethane. The combined organic layer was distilled to obtain an oily residue. The residue was crystallized in methanol to obtain the compound of Formula (VII). (Yield: 44%, HPLC Purity: 99.05%, Any individual maximum impurity: 0.23%, Total Impurity: 0.95%)).

Step-2: Compound of Formula VII-T & Compound of Formula VI

In a 1 L round bottom flask, Chlorotrimethylsilane (66.8 g) was added to a stirring mixture of the compound of Formula (VII) (45 g) and imidazole (55.8 g) in dichloromethane (450 mL) at 0-10° C. in an RBF and stirred at 25-35° C. After the completion of the reaction, water was added and stirred for 30 minutes. The organic layer containing the compound of Formula (VII-T) was separated, cooled to 0 to 10° C. and a solution of pyridine (13.8 g) and p-toluene sulfonic acid monohydrate (29.2 g) in water (36 mL) was added. The reaction mixture was stirred at 25-35° C. After the completion of the reaction, layers were separated, and the organic layer was washed with phosphate buffer and distilled to obtain the compound of Formula (VI) as an oily residue (64 g).

Step-3: Compound of Formula V

In a 1 L round bottom flask, Dimethyl sulfoxide (256 mL) and triethyl amine (44.4 g) were added to a stirring mixture of the compound of Formula (VI) (64 g) in dichloromethane (320 mL) at 0-10° C. Sulfur trioxide-pyridine complex (46.6 g) was added lot-wise and the reaction mixture was stirred at 0 to 10° C. After the completion of reaction, water was added and the reaction mixture was warmed to room temperature. The organic layer was separated and washed with aqueous citric acid solution and concentrated under vacuum below 35° C. to obtain the compound of Formula (V) as a residue (60 g).

Step-4: Compound of Formula IV

In a 1 L round bottom flask, Paraformaldehyde (43.2 g) was added to a stirring mixture of sodium ethoxide (9.7 g) in ethanol (235 mL) and stirred at 50-55° C. A solution of compound of Formula (V) (47 g) in ethanol (470 mL) was added and stirred for 4 hours at 50-55° C. followed by addition of sodium ethoxide (4.8 g) and paraformaldehyde (10.78 g) and stirred at 50-55° C. After completion of the reaction, sodium bisulphite solution was added, stirred for 30 minute and the solvent was removed by distillation under vacuum. The remaining mass was dissolved in MTBE and washed with aqueous sodium bisulphite solution. The MTBE layer was concentrated to get the compound of Formula IV as oily residue. The residue was recrystallized from methanol to obtain the compound of Formula IV. (HPLC purity: 99.99%, Any individual maximum impurity: 0.01%, Total Impurity: 0.01%)

Step-5: Ertugliflozin

In a 500 mL round bottom flaks, the compound of Formula (IV) (15 g) and methane sulphonic acid (75 g) were stirred in dichloromethane (300 mL) for 5 hours at 25-35° C. After the completion of the reaction, the reaction mixture was washed with 1% aqueous sodium bicarbonate solution and the organic layer was distilled off to obtain ertugliflozin.

### Example-2: Amorphous Ertugliflozin

Ertugliflozin (5 g) was stirred in a solvent at specified temperature for 10-60 min to obtain a solution and the solvent was removed to obtain amorphous ertugliflozin as described in the following table—

| Sr. | | | Solvent | |
|---|---|---|---|---|
| No. | Solvent | Temperature | Removal by | Product |
| 1 | ethanol (50 mL) | 55-60° C. | Distillation | 4.8 g |
| 2 | isopropyl | | Distillation at | |
| | alcohol/water | 25-35° C. | reduced pressure | 4.6 g |
| | (1:1) (50 mL) | | | |
| 3 | methanol/water | | Distillation at | |
| | (1:1) (40 mL) | 50-60° C. | reduced pressure | 4.0 g |
| 4 | ethanol (50 mL) | 55-60° C. | Spray Drying | 4.6 g |
| 5 | isopropyl | | Spray drying | 4.5 g |
| | alcohol/ethylene | 70-75° C. | | |
| | glycol (9:1) | | | |
| | (50 mL) | | | |
| 6 | acetone/water | 40-45° C. | Distillation at | 4.6 g |
| | (9:1) (50 mL) | | reduced pressure | |
| 7 | methylene- | | | |
| | dichloride | | | |
| | (50 mL) | 25-30° C. | Distillation | 4.8 g |

### Example-3: Amorphous Solid Dispersion of Ertugliflozin

Ertugliflozin (50 g) and hydroxypropylmethyl cellulose (49 g) were stirred in ethanol (1000 mL) at 25-35° C. for 15-30 min and the solvent was removed at reduced pressure to obtain amorphous solid dispersion of ertugliflozin and hydroxypropylmethyl cellulose.

### Example-4: Amorphous Solid Dispersion of Ertugliflozin

Ertugliflozin (50 g) and co-povidone [a PVP polymer] (52 g) were stirred in methanol (1000 mL) at 25-35° C. for 15-30 min. The resultant mass was passed through a fine filter to remove particulate matter and was subjected to spray drying to obtain amorphous solid dispersion of ertugliflozin and co-povidone. The spray drying was performed under following conditions—

| | Working | Operating |
|---|---|---|
| Parameter | Range | Parameter for Ex-4 |
| Inlet temperature (° C.) | 70-80 | 75 |
| Aspirator % | 60 to 80 | 70-75 |
| Innen gas flow (L/h) | 500 to 550 | 500-520 |
| Pump % | 8 to 12 | 10-11 |
| Feed rate (ml/min) | 6 to 12 | 7.8-8.2 |
| Filter bag pressure (mbar) | 0 to −50 | 0 to −50 |
| Needle tip size (mm) | 0.5 | 0.5 |

These parameters are derived using Buchi mini spray dryer (Inert loop)-B-290. For a different make and/or model of equipment, the parameters may vary.

### Example-5: Amorphous Solid Dispersion of Ertugliflozin

The compound of Formula (IV) (15 g) and methane sulphonic acid (75 g) were stirred in dichloromethane (300 mL) for 5 h at 25-35° C. in an RBF. After reaction completion, the reaction mass was washed with 1% aqueous sodium bicarbonate solution and the organic layer was distilled off and the residue was dissolved in 75 mL ethyl acetate and 6.98 g L-Proline was added. The resulting mass was stirred for 6 h at room temperature and filtered and dried to obtain ertugliflozin-L-proline complex (Yield-94%, HPLC purity 99.99%, Any individual maximum impurity: 0.01%, Total Impurity: 0.01%).

Ertugliflozin-L-Proline complex (15 g) was partitioned between ethyl acetate and water and the organic layer was distilled off. The residue was dissolved in methanol to obtain a clear solution and Co-Povidone (9.8 g) was added and stirred for 30 minutes. The solution was then subjected to spray drying to obtain amorphous solid dispersion of ertugliflozin and co-povidone. (HPLC purity: 99.99%, Any individual maximum impurity: 0.01%, Total Impurity: 0.01%)

### Example-6: Stable Amorphous Ertugliflozin

Amorphous ertugliflozin was stabilized by placing under nitrogen atmosphere in a non-permeable bag and tied with a thread; placing the bag inside a black color polyethylene bag, optionally containing oxygen busters and sealing it; placing the bag inside a triple laminated bag, optionally containing oxygen busters and sealing it; and placing the sealed triple laminated bag inside a high density polyethylene (HDPE) container and sealing it.

### Example-7: Composition of Amorphous Solid Dispersion of Ertugliflozin

Amorphous solid dispersion of ertugliflozin (100 g), mannitol (230 g), pregelatinised starch (35 g) and maize starch (35 g) were blended and wetted with a granulating solution and subsequently granulated. The dried granules were further blended with magnesium stearate and compressed in to tablets.

While the present invention has been described in terms of a few specific aspects, modifications and equivalents thereof, in light of teaching and disclosure of the present invention, which are apparent of the skilled artisan, are to be construed as included within the scope of the invention.

1. An amorphous solid dispersion comprising ertugliflozin.

2. The amorphous solid dispersion according to claim 1, wherein the dispersion comprises ertugliflozin and a polymer.

3. The amorphous solid dispersion according to claim 2, wherein the polymer is selected from a group comprising hydroxypropylmethyl cellulose (HPMC), hydroxypropylmethyl cellulose acetate succinate (HPMC-AS) and polyvinyl pyrrolidone (PVP).

4. The amorphous solid dispersion according to claim 2, wherein the polymer is present in the solid dispersion in a ratio, by weight, from 9:1 to 1:9; from 4:1 to 1:4; from 3:2 to 2:3; or from 1.2:1 to 1:1.2.

5. The amorphous solid dispersion according to claim 1, wherein ertugliflozin has purity of 99.5% or more, having total impurities 0.5% or less and single individual maximum impurity 0.15% or less, as determined by area percentage of HPLC.

6. The amorphous solid dispersion according to claim 1, wherein ertugliflozin has purity of about 99.5% or more, and one or more of compounds selected from A, B, C, D, E, F and G, less than about 0.15% each, as determined by area percentage of HPLC.

7. The amorphous solid dispersion according to claim 1, wherein ertugliflozin does not convert to any other solid form after exposure to 75% RH at 40° C. for at least 3 months.

8. The amorphous solid dispersion according to claim 1, wherein ertugliflozin contains less than 0.5% (wt/wt) of total impurities after exposure to 75% RH at 40° C. for at least 3 months.

9. A process for the preparation of amorphous solid dispersion of ertugliflozin according to claim 1, comprising removal of the solvent from a solution comprising ertugliflozin and a polymer.

10. A process for the preparation amorphous solid dispersion of ertugliflozin comprising:
(a) dissolving ertugliflozin and a polymer in one or more of water, alcohol, glycol, polyol, ketone or halogenated hydrocarbon to obtain a solution; and
(b) removing the solvent from the solution to obtain the amorphous solid dispersion of ertugliflozin.

11. A stable amorphous form of ertugliflozin which does not convert to any other solid form after exposure to 75% RH at 40° C. for at least 3 months.

12. The stable amorphous form of ertugliflozin according to claim 11, wherein ertugliflozin has purity of 99.5% or more, having total impurities 0.5% or less and single individual maximum impurity 0.15% or less, as determined by area percentage of HPLC.

13. A pharmaceutical composition comprising stable amorphous form of ertugliflozin according to claim 11, and pharmaceutically acceptable carrier.

14. A composition comprising amorphous solid dispersion of ertugliflozin according to claim 1 and a pharmaceutically acceptable carrier.

15. A pharmaceutical composition comprising amorphous solid dispersion of ertugliflozin according to claim 1 and one or more pharmaceutically acceptable carriers, excipients or diluents.
